(19) United States
(12) Patent Application Publication
(10) Pub. No.: US 2024/0081751 A1
(43) Pub. Date: Mar. 14, 2024

(54) SYSTEM AND METHOD FOR MANAGING CARDIOVASCULAR RISK IN BREAST CANCER PATIENTS

(71) Applicants: Alexandra Murphy, Heidelberg (AU); Matias Yudi, Heidelberg (AU)

(72) Inventors: Alexandra Murphy, Heidelberg (AU); Matias Yudi, Heidelberg (AU)

(21) Appl. No.: 18/300,914

(22) Filed: Apr. 14, 2023

Related U.S. Application Data
(60) Provisional application No. 63/405,717, filed on Sep. 12, 2022.

(51) Int. Cl. A61B 5/00; G06F 3/0482; G06F 3/04847

(52) U.S. Cl. CPC A61B 5/7445; A61B 5/7275; G06F 3/0482; G06F 3/04847

(57) ABSTRACT

A system and method for reducing cardiovascular risks in breast cancer patients. The method includes providing an interface on a user device for receiving details of the user, such as details of the therapy, medical conditions of the user, blood pressure, and cholesterol levels. The system can determine the current fitness level of the user. Based on the received details, the system can determine cardiovascular risk factors for the user. The cardiovascular risk factors can be presented through a dashboard screen on the user device.

### CROSS-REFERENCE TO RELATED APPLICATIONS

This application claims priority from of a U.S. Patent Application Ser. No. 63/405,717, filed on Sep. 12, 2022, the disclosures of which is hereby incorporated herein by reference in their entirety.

### FIELD OF INVENTION

The present invention relates to a system and method for improving the survival rate in breast cancer patients, and more particularly, the present invention relates to a system and method for reducing cardiovascular risks in breast cancer patients.

### BACKGROUND

Breast cancer is the first or second most common cancer diagnosed in women in most parts of the world. The rapid increase in awareness about breast cancer and the advancements in therapies have sharply improved the survival rate. However, patients with breast cancer or patients recovering from breast cancer suffer from cardiovascular diseases which have become a major cause of poor recovery or mortality. The frequency of patients recovering from breast cancer is increasing but the frequency of occurrence of cardiovascular diseases in such patients is rapidly rising. The same has also been termed a Cardio-Oncology-epidemic. However, the risk of cardiovascular diseases in breast cancer patients can be significantly reduced by increased awareness about cardiovascular diseases and health management.

Smartphone apps that allow users to manage their exercise routines, food habits, medications, and the like are well known. However, a need is there for a specialized application that targets cardiovascular risks in breast cancer patients.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a system and method for managing lifestyle in breast cancer patients to reduce the risk of cardiovascular diseases.

It is another object of the present invention that the system and method promote prescribed exercises in breast cancer patients.

It is still another object of the present invention that the system and method allow tracking of cardiovascular risk factors.

It is yet another object of the present invention that the system and method provide for the assessment of dietary habits.

It is a further object of the present invention that the system and method allow for interactive and personalized feedback.

It is still a further object of the present invention that the system and method allow for community engagement.

In one aspect, disclosed are a system and method for managing lifestyle including the health of breast cancer patients such as reducing the risk of cardiovascular diseases.

In one aspect, disclosed is a method for reducing cardiovascular risks in breast cancer patients, the method implemented within a system comprising a processor and a memory, the method comprising presenting, by the system, a first screen on a user device for receiving detail of therapy; determining, by the system, through one or more sensors of the user device, current fitness level of the user, wherein the system presents a second screen on the user device, wherein the second screen comprises a soft button to start a test; receiving, through a third screen on the user device, medical conditions of the user, blood pressure, and cholesterol levels; determining, by the system, by processing cardiovascular risk factors of the user based on the medical conditions of the user, blood pressure, cholesterol levels, and the current fitness level of the user; and presenting, a dashboard screen on the user device, wherein the dashboard screen comprises the cardiovascular risk factors. The first screen comprises a list of therapies, a slider button associated with each item in the list of the therapies, wherein the method further comprises selecting one or more items from the list of therapies through the respective slider buttons for the detail of therapy. The one or more sensors comprises accelerometer. The current fitness level of the user is based on a number of steps taken and a distance walked by the user as a function of time. The method further comprises grouping the cardiovascular risk factors into high risk factors, suboptimal parameters, and on-target parameters, wherein the high-risk factors are presented in red on the dashboard screen, the on-target parameters are presented in green on the dashboard screen, and suboptimal parameters are presented in yellow on the dashboard screen. The method further comprises presenting, by the system, on a screen, relevant activities for the user; and tracking, by the system, progress of the user in performing the relevant activities. The method further comprises providing an interface for telehealth consultation.

### BRIEF DESCRIPTION OF THE DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a block diagram showing an environmental view of the system, according to an exemplary embodiment of the present invention.

FIG. 2 is a block diagram showing the architecture of the system, according to an exemplary embodiment of the present invention.

FIG. 3 shows a screen of the interface provided by the system, according to an exemplary embodiment of the present invention.

FIG. 4 shows a screen of an interface, according to an exemplary embodiment of the present invention.

FIG. 5 shows another screen of the interface, according to an exemplary embodiment of the present invention.

FIG. 6 shows another screen of the interface, according to an exemplary embodiment of the present invention.

FIG. 7 shows another screen of the interface, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, the reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting of embodiments of the invention. As used herein, the singular forms “a”, “an”, and “the” are intended to include the plural forms as well, unless the context indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed are a system and method for managing the lifestyle of breast cancer patients to reduce cardiovascular diseases. The disclosed system particularly emphasizes physical activity to help patients remain healthy and prevents the development of cardiovascular risk factors. However, the disclosed system aims at incorporating all core aspects of cardiovascular risk reduction. It was observed that independent methods for exercise promotion and dietary management do not provide any good results in managing cardiovascular risks in breast cancer patients. The disclosed system aims at providing an ecosystem for improving the lifestyle and health of breast cancer patients emphasizing the cardiovascular risk factors.

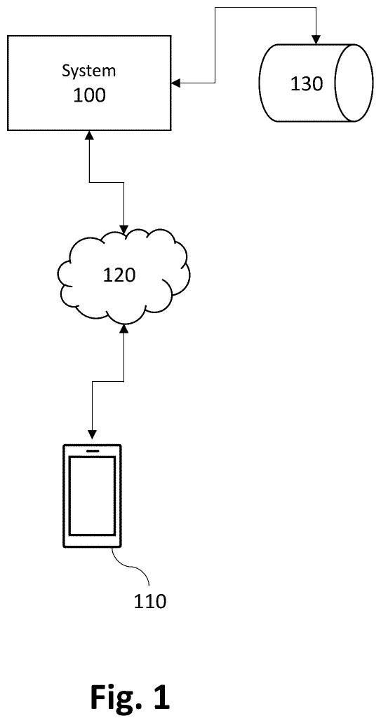

Referring to FIG. 1 is a block diagram showing an environment of the disclosed system 100. The disclosed system can interact with one or more user devices 110 through a network 120. The user devices can be computer devices with network connectivity, and examples of the user devices may include a smartphone, laptop, desktop, tablet computer, personal digital assistant (“PDA”), and smartwatches. Smartwatches can also provide various monitored health parameters for the user. The user device can include an operating system, such as but not limited to Android, iOS, Windows, macOS, blackberry, and Linux. The user device can have an input for receiving an input and a display for output so that a user can interact with the disclosed system 100.

The network may be a wired network, a wireless network, or may include a combination of wired and wireless networks. For example, network 120 may be a local area network (LAN), a wide area network (WAN), a wireless WAN, a wireless LAN (WLAN), a metropolitan area network (MAN), a wireless MAN network, a cellular data network, a cellular voice network, the Internet, etc. FIG. 1 shows a single device connected to the system is for illustration only, however, it is obvious that multiple user devices can connect with the disclosed system at various times and at also at the same time. Also, FIG. 1 shows a single network; however, it is obvious that different devices can connect with the disclosed system through different networks and a single-user device can connect through more than one network. The disclosed system can operably couple to a database management system 130. Database management system 130 can be used to store data, such as profiles of the users.

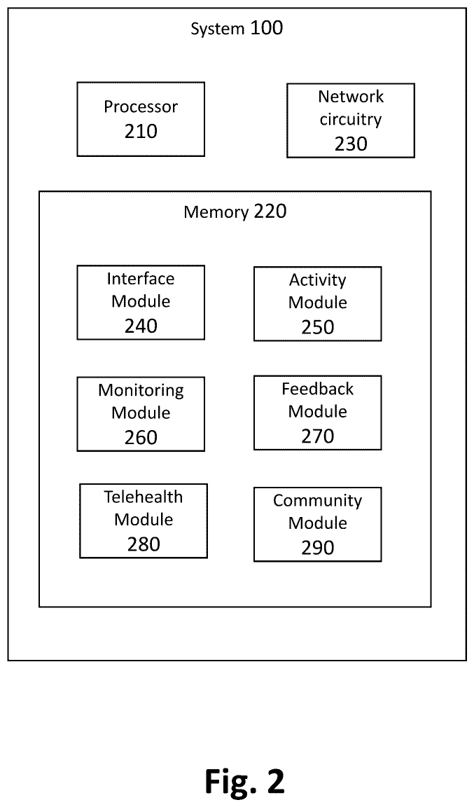

Referring to FIG. 2 is a block diagram showing the architecture of the disclosed system 100. The system 100 includes a processor 210 and a memory 220. The processor is operably coupled to the memory. The processor can be any logic circuitry that responds to, and processes instructions fetched from the memory. The memory may include one or more memory chips capable of storing data and allowing any storage location to be directly accessed by the processor. The memory includes modules according to the present invention for execution by the processor to perform one or more steps of the disclosed methodology. System 100 can also include a network circuitry 230 connecting to an external network.

The memory can include an interface module 240, an activity module 250, a monitoring module 260, a feedback module 270, a telehealth module 280, and a community module 290. The interface module upon execution by the processor can provide an interface on the user device. The activity module upon execution by the processor can provide prescribed activities for the user and track said activities. The monitoring module upon execution by the processor can track cardiovascular risk factors. The feedback module upon execution by the processor can provide personalized messages, instructions, and feedback on progress to the user. The telehealth module upon execution by the processor can provide for online/remote medical consultations. The community module upon execution by the processor can provide for engaging the users on social platforms and communities.

The disclosed system can be implemented on one or more servers. One or more servers include cloud servers. One or more servers can be at a single location or can be geographically dispersed. Also, FIG. 1 shows the different modules executed by the disclosed system, however, one or more steps of the disclosed methods can be performed in the user devices and executed by the processor of the user devices without departing from the scope of the present invention.

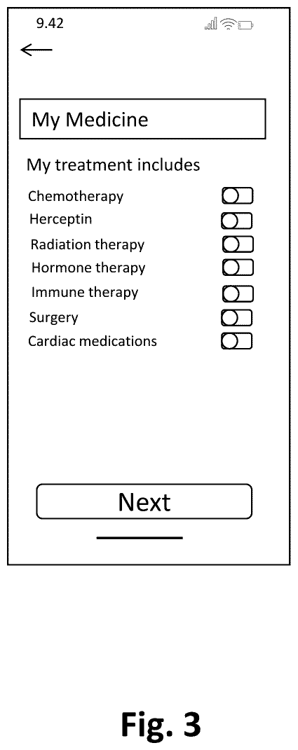

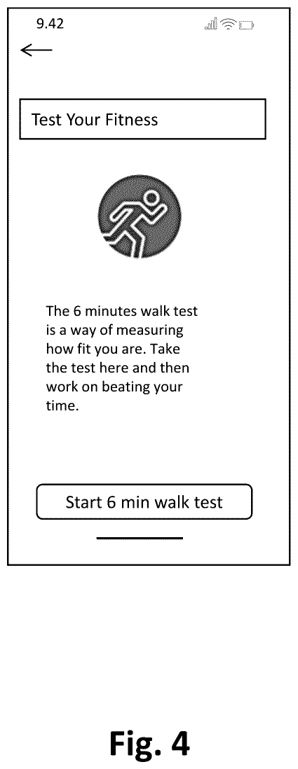

The interface module upon execution by the processor can provide an interface that allows a user to interact with the disclosed system wherein information can be provided by the user to the system through the user interface and information can be presented to the user by the system through the user interface. The interface can be provided as application software that can be installed on the user device. The application software can be developed for Android™, iOS, and any other known operating platform for mobile devices. The application software can be made available through a distribution service provider, for example, Google Play™ operated and developed by Google, and the app store by Apple. In addition to the application software, a website-based interface can also be provided through the world-wide-web. The application software can also be provided for the desktop environment, such as Windows™, Linux, and macOS. FIGS. 3-7 shows various screens of the interface.

FIG. 3 shows a screen through which details of therapy being received by the user for breast cancer can be received by the system. A list of therapies can be included and each item in the list has an associated slider button. The slider button can be used to select an item in the list. The user can select his prescribed therapies from the list and can then click the next button at bottom of the screen. The selection of therapies can be received by the system and stored in the user profile as a record. Through the second screen, the system can determine the current fitness level of the user. The screen shows a 6-minute walk test, wherein the user can click the start button at the bottom of the screen to start the test. The system can determine the fitness level using a variety of health parameters. At least the system can determine the number of steps taken and the distance walked by the user as a function of time to determine the fitness level of the user. It is understood that the user device can support monitoring such parameters i.e., steps taken by the user. For example, the smartphone is equipped with an accelerometer and sensors that allow estimating the number of steps taken by the user. Different parameters can be tracked by the disclosed system, for example, using the smartphone, wearable health band, and the like, and any such parameter is within the scope of the present invention for estimating the fitness level of the user. It is obvious, that the fitness level as estimated may not be precise but within a medically acceptable range of accuracy.

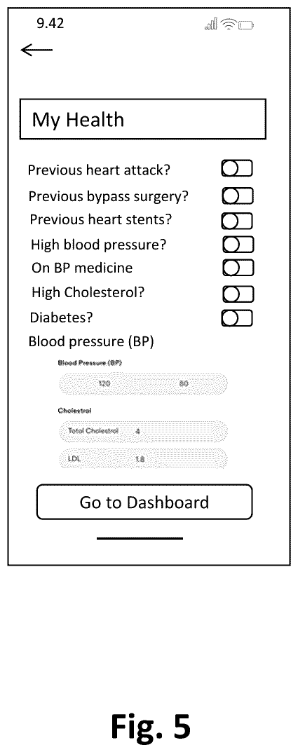

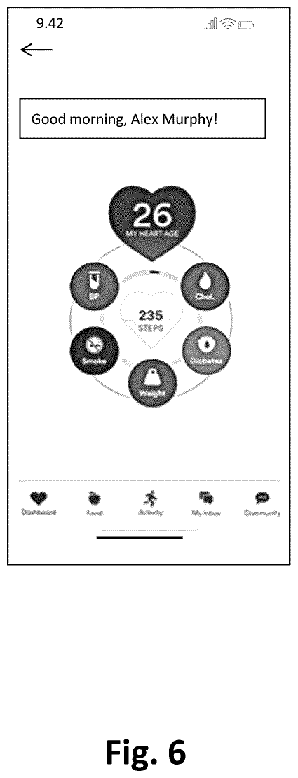

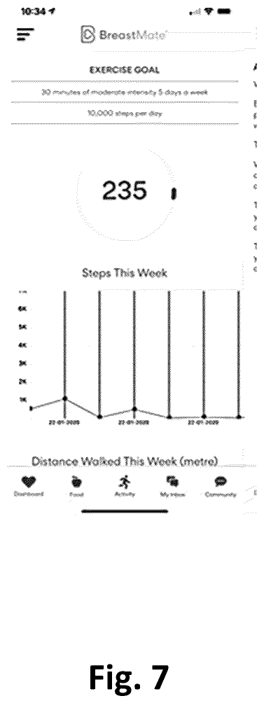

Through the third screen, shown in FIG. 5, the system can receive other medical conditions of the user, blood pressure, and cholesterol levels of the user. The user can then go to the dashboard screen using the button at bottom of the screen. The dashboard screen is shown in FIG. 6. Through the dashboard screen, the system can be provided with a comprehensive assessment of cardiovascular risk factors and utilizes an automated ‘traffic light’ system to represent target achievement. ‘High Risk’ parameters can be displayed in red, ‘Suboptimal’ parameters can be displayed in yellow, and ‘On Target’ parameters can be displayed in green. The users can receive a notification if a risk factor parameter is not on target. Through the screen shown in FIG. 7, the exercise goals and progress of the user can be presented by the system.

The disclosed system can also provide a peer-to-peer networking platform for patients with breast cancer. Through the community module, the disclosed system can suggest social groups to the user and allow the user to engage with the group. Also, the community module may allow the user to post on a community forum, wherein the user can post questions, messages, and the like. The user can share their progress with other users, such as steps taken on the day. The user can also check updates from other users and can also answer questions posted in the community forum or board.

The activity module upon execution by the processor can provide relevant activities for the user and track the progress of prescribed activities performed by the user. This can be an important step of the disclosed method as the exercise can directly help maintain the cardiovascular health of the user. The activity module can present real-time feedback to the user for their activity. The activity module can monitor the activity of the user through different sensors, such as an accelerometer of the smartphone can be used to estimate the number of steps taken and the distance walked. The activity module can present exercise goals to the user and also show the progress through the interface on the user device. For example, FIG. 7 shows the exercise goals as set out by the activity module and the progress of the user per day, per week, and, per month. The activity module can take data from different sensors, such as those in the smartphone and fitness bands.

The monitoring module can receive the health condition of the users in terms of different health parameters, such as cholesterol, blood pressure, smoking frequency, blood glucose levels, cholesterol, and the like. Different parameters that can affect cardiovascular health can be monitored by the system. Such parameters and their current values can be presented in an interactive manner on a dashboard screen, as shown in FIG. 6. Values of many of the parameters can be received from the user through the interface, wherein the user can update the value anytime. For example, the user can update the cholesterol level when tested again. Many of the parameters can be automatically received by the system. For example, the disclosed system may receive current blood sugar levels from a testing device when the user checks their sugar levels. The activity module can notify the user when the value of any parameter is out of desired range, at alarming levels, or in any defined condition. As described above, different color levels can be used to show the status of different parameters, wherein red indicates alarming levels.

The feedback module can intelligently provide for interaction with the user by sending personalized messages through the interface. Such messages may provide useful tips, instructions, and the like to users for managing a healthy lifestyle. Moreover, messages to motivate the user based on their progress in different activities can be generated by the system. The system can use artificial intelligence features to track activities and generate personalized messages for the user. The system aims at increasing confidence and motivation to exercise while overcoming barriers to being physically active.

The telehealth module allows for video consultation through the interface with medical professionals, coaches, and the like. The disclosed system can show the progress of the user and different health parameters to the medical professional. It is to be noted that the telehealth module may obtain consent from the user before sharing any information. The feature can be advantageous in any urgent situation and when the user cannot visit for a consultation. The telehealth module may help overcome geographic barriers for patients living in rural and remote areas.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for reducing cardiovascular risks in breast cancer patients, the method implemented within a system comprising a processor and a memory, the method comprising:
presenting, by the system, a first screen on a user device for receiving detail of therapy;
determining, by the system, through one or more sensors of the user device, current fitness level of the user, wherein the system presents a second screen on the user device, wherein the second screen comprises a soft button to start a test;
receiving, through a third screen on the user device, medical conditions of the user, blood pressure, and cholesterol levels;
determining, by the system, by processing cardiovascular risk factors of the user based on the medical conditions of the user, blood pressure, cholesterol levels, and the current fitness level of the user; and
presenting, a dashboard screen on the user device, wherein the dashboard screen comprises the cardiovascular risk factors.

2. The method according to claim 1, wherein the first screen comprises a list of therapies, a slider button associated with each item in the list of the therapies, wherein the method further comprises:
selecting one or more items from the list of therapies through the respective slider buttons for the detail of therapy.

3. The method according to claim 1, wherein the one or more sensors comprise an accelerometer.

4. The method according to claim 1, wherein the current fitness level of the user is based on a number of steps taken and a distance walked by the user as a function of time.

5. The method according to claim 1, wherein the method further comprises:
grouping the cardiovascular risk factors into high risk factors, suboptimal parameters, and on-target parameters, wherein the high-risk factors are presented in red on the dashboard screen, the on-target parameters are presented in green on the dashboard screen, and suboptimal parameters are presented in yellow on the dashboard screen.

6. The method according to claim 1, wherein the method further comprises:
presenting, by the system, on a screen, relevant activities for the user; and
tracking, by the system, a progress of the user in performing the relevant activities.

7. The method according to claim 1, wherein the method further comprises:
providing an interface for telehealth consultation.

8. A system for reducing cardiovascular risks in breast cancer patients, the system comprising a processor and a memory, the system configured to implement a method comprising:
presenting, by the system, a first screen on a user device for receiving detail of therapy;
determining, by the system, through one or more sensors of the user device, current fitness level of the user, wherein the system presents a second screen on the user device, wherein the second screen comprises a soft button to start a test;
receiving, through a third screen on the user device, medical conditions of the user, blood pressure, and cholesterol levels;
determining, by the system, by processing cardiovascular risk factors of the user based on the medical conditions of the user, blood pressure, cholesterol levels, and the current fitness level of the user; and
presenting, a dashboard screen on the user device, wherein the dashboard screen comprises the cardiovascular risk factors.

9. The system according to claim 8, wherein the first screen comprises a list of therapies, a slider button associated with each item in the list of the therapies, wherein the method further comprises:
selecting one or more items from the list of therapies through the respective slider buttons for the detail of therapy.

10. The system according to claim 8, wherein the one or more sensors comprises accelerometer.

11. The system according to claim 8, wherein the current fitness level of the user is based on a number of steps taken and a distance walked by the user as a function of time.

12. The system according to claim 8, wherein the method further comprises:
grouping the cardiovascular risk factors into high risk factors, suboptimal parameters, and on-target parameters, wherein the high-risk factors are presented in red on the dashboard screen, the on-target parameters are presented in green on the dashboard screen, and suboptimal parameters are presented in yellow on the dashboard screen.

13. The system according to claim 8, wherein the method further comprises:
presenting, by the system, on a screen, relevant activities for the user; and
tracking, by the system, progress of the user in performing the relevant activities.

14. The system according to claim 8, wherein the method further comprises:
providing an interface for telehealth consultation.
